(19) United States
(12) Patent Application Publication
(10) Pub. No.: US 2010/0297744 A1
(43) Pub. Date: Nov. 25, 2010

(54) Multi-Channel Bioreactor with the Immobillization of Optical Sensing Membrane

(75) Inventors: Jong IL Rhee, Gwangju (KR); Ok-Jae Sohn, Jeollanam-do (KR)

Correspondence Address: THE WEBB LAW FIRM, P.C., 700 KOPPERS BUILDING, 436 SEVENTH AVENUE, PITTSBURGH, PA, 15219, US

(73) Assignee: INDUSTRY FOUNDATION OF CHONNAM NATIONAL UNIVERSITY, Gwangju (KR)

(21) Appl. No.: 12/671,978

(22) PCT Filed: Dec. 26, 2007
(86) PCT No.: PCT/KR07/06822

§ 371 Date: Feb. 3, 2010

(30) Foreign Application Priority Data
Aug. 8, 2007 (KR) 10-2007-0079549

(51) Int. Cl. C12M 1/34

(52) U.S. Cl. 435/287.2; 435/288.7

(57) ABSTRACT

The present invention relates to a multi-channel microbioreactor. More specifically, the present invention relates to a multi-channel microbioreactor having a number of wells in which an optical sensing membrane comprising a fluorescent dye and a bioconjugate, or a sensor material including a biomolecule conjugated to a fluorescent dye and a bioconjugate is formed. The multi-channel microbioreactor according to the present invention can be used for an in-situ optical detection of dissolved oxygen, carbon dioxide, pH, monosaccharides, polysaccharides, organic acids, alcohols, cholesterol, choline and xanthine, etc.

### TECHNICAL FIELD

The present invention relates to a multi-channel micro bioreactor for an optical detection which can be used for on-line monitoring of a biological process.

### BACKGROUND ART

The present invention relates to a multi-channel microbioreactor. More specifically, the present invention relates to a multi-channel micro bioreactor which can be used for an in-situ optical detection of dissolved oxygen, carbon dioxide, pH, monosaccharides, polysaccharides, organic acids, alcohols, cholesterol, choline and xanthine, etc., wherein a number of wells in which an optical sensing membrane comprising a fluorescent dye and a bioconjugate, or a sensor material including a biomolecule conjugated to a fluorescent dye and a bioconjugate is formed.

For biological processes, quick and accurate analysis of information is essential for evaluating rapidly a relationship between a biological system and the biological processes and for subsequently optimizing the biological processes. Especially, a sensor technology based on optical techniques has been used for a non-invading and continuous analysis. In addition, it has been used for on-line monitoring of various industrial processes (pharmaceutical, food, and environmental industries, etc.) and also for developing a technology for monitoring biological processes, which tremendously contribute to modern biological sciences. Until now, no studies have been made in Korea regarding a micro array biological process system based on an optical sensor technology. On the other hand, in other developed countries including the United States and Germany, it is getting more attention and many studies are currently being made. Moreover, some of research institutes or companies are diligently working to commercialize it.

In order to obtain real time information about biological or chemical processes as much as possible, a high-throughput processor is now actively being developed. Recently, a technology for a quantitative or qualitative analysis of large-amount sample is developed, thanks to the control of micro fluid and development in nano-technology. However, the present technology has a limitation in that real time obtainment of information about living cells, in particular cells contained in a bioreactor, is difficult.

Generally, in order to obtain real time information about growth state of living cells, a monitoring method using an electrode type sensor that is attached to a large-size fermentation apparatus or a method of analyzing a sample outside a reactor after sampling has been used. For general research works and developments related to a biological process technology, a large-volume (i.e. more than 100 mL) shaker incubator or biological reactor is generally used. A shaker incubator is typically used for basic studies and developments of biological processes, easy to operate and can be used for search of new microbial strains but difficult for on-line monitoring and control, so it is dependent on an off-line analysis. A commercial biological fermentation apparatus usually has a volume more than one liter and is used for mass production of biological products. Because various electrode-type sensors can be attached to a biological fermentation apparatus, it can be conveniently used for on-line monitoring and control of the processes. However, due to a big size of an apparatus, it is disadvantageous in that a tremendous amount of time and labor forces are required and a great amount of an expensive substrate can be wasted. Furthermore, for an analysis of a sample which cannot be detected using an electrode-type sensor, lots of research labor forces are required and analytical cost can be high, resulting in an unnecessary waste. In particular, it is disadvantageous in that it cannot be used for an obtainment of real time information about cell growth. Therefore, in order to solve the problems associated with a conventional shaker incubator and a biological reactor, a mini size, multi-biological process system which can be easily controlled and used for on-line monitoring needs to be developed.

### DISCLOSURE OF INVENTION

### Technical Subject

In order to solve the problems described in the above, a biological reaction is carried out by using a micro bioreactor according to the present invention, provided that an optimum condition for the biological reaction is established by fast analysis of the reaction via on-line monitoring and a multi-channel analysis is carried out by using a single bioreactor. Thus, the object of the present invention is to provide a multi-channel micro bioreactor which can be used for saving research labor forces and cost required for an individual analysis.

### Technical Solution

In order to achieve the object of the present invention described in the above, the present invention provides a multi-channel micro bioreactor having a number of wells, in which an optical sensing membrane comprising a fluorescent dye and a bioconjugate, or a sensor material including a biomolecule conjugated to a fluorescent dye and a bioconjugate is formed.

Wells of the multi-channel micro bioreactor according to the present invention can have any shape including a pillar, a cylinder, a diamond-shaped pillar and a test tube, etc. as long as it can contain a liquid. Wells having a shape of a pillar or a cylinder with flat bottom are preferable for an easy optical detection and an even coating of optical sensing membrane.

For a multi-channel micro bioreactor having a number of wells, a microtiter plate, a test tube or a rack to which probe is immobilized can be used. It is preferable to use a microtiter plate having 4 to 1,536 wells in terms of easy installment of the bioreactor into a system and even immobilization.

The multi-channel micro bioreactor according to the present invention is characterized in that it comprises a number of wells formed in a single frame, wherein said wells are aligned to obtain an efficient detection by the system, said formed and aligned wells include an optical sensing membrane formed at the bottom of each well, said optical sensing membrane formed at the bottom of the wells is a single membrane or divided into number n, denoting an integer between 1 and 100, each inclusive, and said optical membrane is individually formed depending on each well. Said optical sensing membrane may contain a single or more than one sensor material to simultaneously detect various materials. Said optical sensing membrane includes each of sensor materials, that are adsorbed, bonded covalently, or captured by particles such as nano particles and micro particles, etc.

As a sensor material comprised in the multi-channel micro bioreactor according to the present invention, a fluorescent dye and a bioconjugate, or a biomolecule conjugated to a fluorescent dye and bioconjugate can be used to carry out an on-line monitoring of dissolved oxygen, carbon dioxide, pH, monosaccharides, polysaccharides, organic acids, alcohols, cholesterol, choline and xanthine, etc. contained in the bioreactor based on an optical detection method.

For a fluorescent dye, it is selected from a group consisting of a ruthenium complex, HPTS (8-hydroxypyrene-1,3,6-trisulfonate trisodium salt) and fluorescein amine. Complexes based on ruthenium have a characteristic fluorescent activity at certain wavelengths. For example, RuDPP (tris(4,7-diphenyl-1,10-phenanthroline)ruthenium (II) complex) can characteristically emit fluorescence at the wavelength of 580 nm when it is excited at the wavelength of 470 nm. Its fluorescence intensity is inversely proportional to a concentration of oxygen. HPTS is also used as a fluorescent dye to detect carbon dioxide. Fluorescein amine is a substance that is used as a fluorescent dye for measuring pH.

HTPS, which is used as a fluorescent dye in the present invention, requires a polymer membrane that is selectively permeable to carbon dioxide in an aqueous solution. The reason is that, since the fluorescence intensity of HPTS varies according not only to the concentration of carbon dioxide but also to a change in pH and concentration of hydrogen ion, when gel is formed to immobilize HPTS by sol-gel method, ethanol, that is used as a solvent, becomes evaporated to form a multi-porous gel (Xerogel) having a three-dimensional network structure and a number of silanol groups are formed on the surface of said multi-porous gel to give a hydrophilic gel, which prohibits the penetration of hydrogen ions to the sensing membrane, thus eventually causing a problem for the detection of carbon dioxide. Therefore, in the present invention, a polymer material that is selected from a group consisting of a silicone resin, PMMA and vernice, that are all hydrophobic polymers, or a mixture thereof is used to form a membrane selectively permeable to carbon dioxide on top of the HPTS sensor membrane, in order to achieve a selective detection of dissolved carbon dioxide. For said hydrophobic polymer, PMMA is preferably used to obtain stabilization of fluorescent signals.

For the above-described bioconjugate, quantum dots having CdSe core that is coated with ZnS shell are coated with a hydrophilic surfactant and then conjugated with oxidases or oxidases and peroxidases. The oxidases used for said bioconjugate are preferably selected from a group consisting of glucose oxidase (GOD), lactate oxidase (LOD), tyramine oxidase (TOD), ascorbic acid oxidase, and xanthine oxidase or a mixture thereof. Said hydrophilic surfactants are preferably selected from a group consisting of mercaptopropionic acid (MPA), mercaptoacetic acid (MAA), mercaptosuccinic acid (MSA), dithiothreitol (DTT), glutathione, histidine, and thiol-containing silanes or a mixture thereof. In particular, said peroxidase is preferably horseradish peroxidase (HRP).

The above described fluorescent dye and bioconjugate can form a sensor material when they are used together with a biomolecule such as protein, amino acid, enzyme, antigen and antibody, etc. Reaction of an analyte with said biomolecule would affect the concentration of dissolved oxygen and carbon dioxide, and pH, resulting in a change in fluorescence intensity that can be used for an identification of the analyte and the determination of its concentration. For example, when an analyte reacts with oxygen and an oxidative enzyme, an oxidized product and hydrogen peroxide will be obtained, causing a reduction in oxygen concentration.

As such, according to the present invention, dissolved oxygen, carbon dioxide, pH, monosaccharides, polysaccharides, organic acids, alcohols, cholesterol, choline and xanthine, etc., can be detected based on an optical detection method by using a sensor material including a fluorescent dye and a bioconjugate or a biomolecule conjugated to a fluorescent dye and a bioconjugate.

Meanwhile, for the immobilization of said sensor material, chemically inert and physically stable sol-gel method is used to form a permeable material. Covalent bond between an epoxy group of sol-gel and an amine group of a biomolecule inhibits the loss of the biomolecules under washing condition. Further, it allows the sensing membrane to maintain its high sensitivity. A typical characteristic of sol-gel which is applied for encapsulation of organic and biological materials or immobilization of biomolecules significantly contributes to the stability and the sensitivity of the sensing membrane for detecting glucose, lactic acid and tyramine. For a material used for said sol-gel method, alkoxysilane is employed, and example thereof includes 3-glycidoxypropyltrimethoxysilane (GPTMS), methyltriethoxysilane (MTES), aminopropyltrimethoxysilane (APTMS), phenyltrimethoxysilane (PTMS), methyltrimethoxysilane (MTMS) or a mixture thereof.

For carrying out a sol-gel method, alkoxysilane can be used either alone or in a mixture with others. When a fluorescent material and a bioconjugate are used as a transducer and a biological molecule is employed as a biological detection element, a change in mixing ratio of alkoxysilane in an alkoxysilane mixture can yield varying properties including different response speed, etc. of a sensing membrane used for the detection of the concentration of glucose, lactic acid and tyramine. Therefore, for the immobilization of fluorescent dye and a bioconjugate, GPTMS and MTES are preferably comprised in a volume ratio of 1:1-2, especially 1:2. For the immobilization of a biomolecule, GPTMS and APTMS are preferably comprised in a volume ratio of 2-4:1, especially 4:1. For a sol-gel condensation reaction, 35% hydrochloric acid as an acid catalyst or tetramethylammoniumhydroxide (TMAOH) as a base catalyst and water are used. High-purity ethanol is used as a solvent.

There are two ways that can be used for the immobilization of the sensor material of the present invention to a number of wells: i.e., firstly, an optical sensing membrane is directly formed to the wells by using a sol-gel method, and secondly, a transparent polymer film having high light transmittance such as PET, PC, PES, PAR and PP, etc. or a silicone resin film is coated with an optical sensing membrane comprising a sensor material by using sol-gel method and then the resulting film is immobilized using an adhesive.

The immobilized optical sensing membrane described in the above can be divided into n sections (n denotes an integer between 1 and 100, each inclusive) and a separate sensing material can be included in each divided membrane to achieve an individual optical detection of number n for a single well. As an example of said divided optical sensing membrane, a detection result obtained from the optical sensing membrane divided into four sections is described in FIG. 9.

Wells of the multi-channel micro bioreactor according to the present invention are characterized in that a baffle board is installed on the cover of the well. When an analyte is introduced into the bioreactor of the present invention and then stirred, the analyte with inertia force will move in certain direction until it receives a resistance from the baffle board. As a result, the analyte will drastically change its course and a stirring effect of the analyte, an oxygen-transferring speed and a material-transferring speed will increase. For said baffle board, any board in a form of plate, stick or rod can be used. The size of a baffle board is not limited as long as the analyte can be easily stirred. The volume of the baffle board is preferably 1-50% of the total volume of the well. In addition, a number of baffle boards can be installed over a single well as occasion demands.

### BRIEF DESCRIPTION OF DRAWINGS

FIG. 1 is a diagram showing the multi-channel microbioreactor.

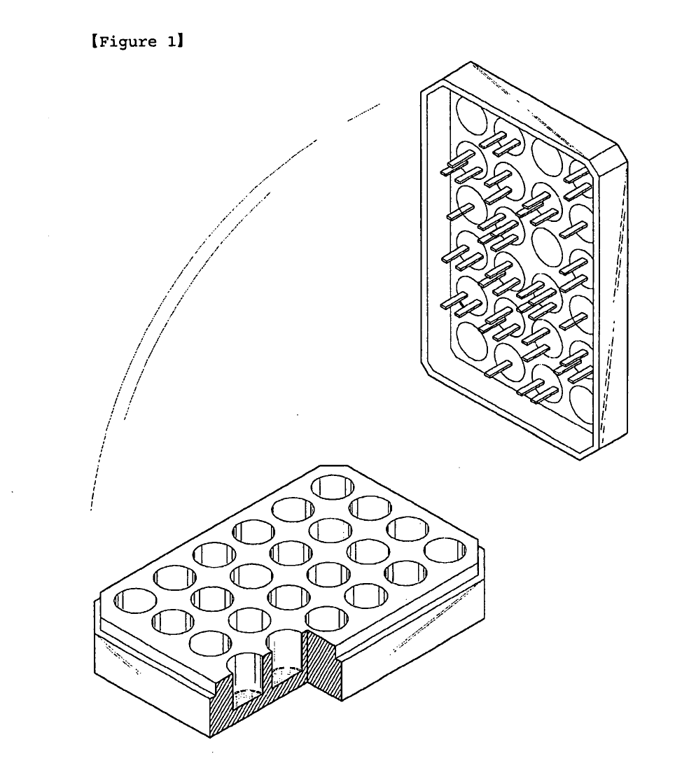

FIG. 2 is a linear graph showing the detection of dissolved oxygen by using the sensing membrane.

FIG. 3 shows a result obtained from the selective detection of carbon dioxide molecules using PMMA.

FIG. 4 is a linear graph showing the measurement result using the optical pH sensing membrane.

FIG. 5 is a result obtained from the on-line monitoring of dissolved oxygen during E. coliBL21 culture.

FIG. 6 is a result obtained from the monitoring of a change in dissolved oxygen in the culture solution to which E. coli has not been inoculated (control group).

FIG. 7 is a result obtained from the on-line monitoring of dissolved oxygen during yeast culture.

FIG. 8 is a result obtained from the on-line monitoring of dissolved oxygen during Bascillus culture.

FIG. 9 shows a detection result of fluorescence intensity in microtiter plate wherein individual optical sensing membranes are divided into several sections.

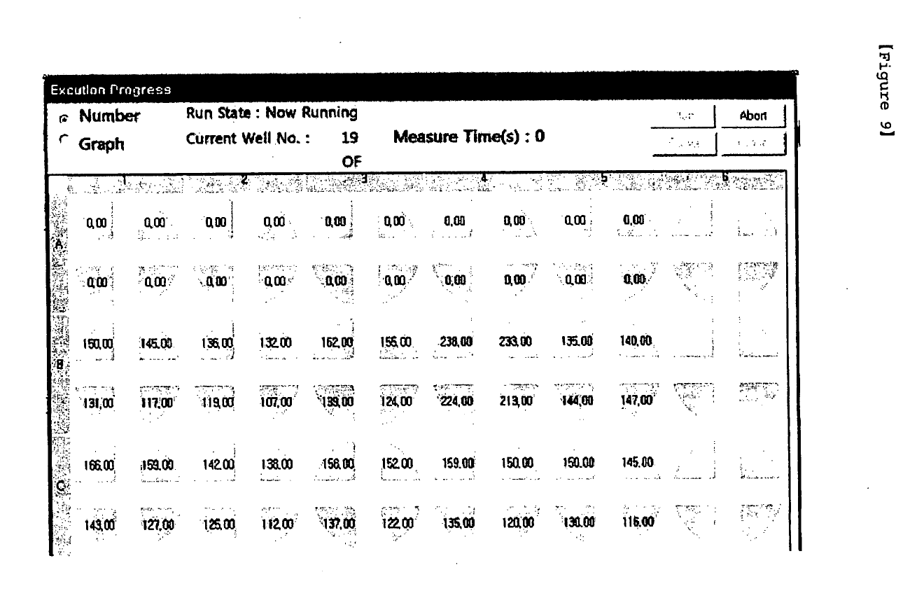

### BEST MODE FOR CARRYING OUT THE INVENTION

Hereinafter, the present invention is described in more detail based on the following examples. However, it is only to specifically exemplify the present invention and in no case the scope of the present invention is limited by these examples.

### Example 1

### Immobilization of Ruthenium Complex

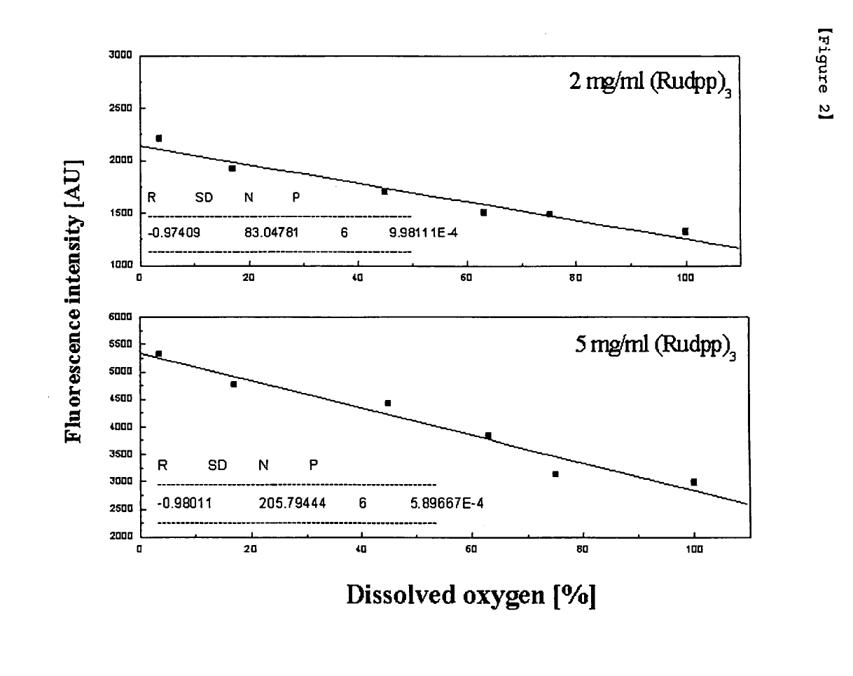

In order to immobilize a ruthenium complex to the wells of a microtiter plate, tetraorthosilicate (98%, 0.6 ml) and methyltrimethoxysilane (MTMS; 98%, 0.6 ml) were admixed to each other and maintained under nitrogen gas. A solution of tetraethylammoniumhydroxide (25%, 0.4 ml) was mixed with highly pure ethanol (1.5 ml) to give a mixture, which was then cooled on ice. After mixing this solution with the above solution maintained under nitrogen gas, the resultant was stirred for 3 hrs at room temperature to give a sol-gel solution. RuDPP complex (tris(4,7-diphenyl-1,10-phenanthroline)ruthenium (II) complex), which had been dissolved in highly pure ethanol to the concentrations of 2 mg/mL and 5 mg/mL each, was mixed with said sol-gel solution in a mixing ratio of 1:1. After stirring the resulting mixture at room temperature for one day, it was coated onto the bottom surface of the wells. Thereafter, the coated microtiter plate was air-dried at room temperature for five days and further dried at 80° C. for two days for the immobilization. The amount of dissolved oxygen was determined for the groups wherein RuDPP was comprised in an amount of 2 mg/mL or 5 mg/mL, respectively, and the results are shown in FIG. 2. As a result, it was found that the sensing membrane comprising RuDPP at a concentration of 5 mg/mL showed better performance (difference in the amount of dissolved oxygen between 0% and 100%) as having steeper slope and better sensitivity. In addition, the linearity for the sensing membrane comprising 5 mg/mL of RuDPP was −0.98011, indicating that it is based on more precise measurement compared to the sensing membrane comprising 2 mg/mL of RuDPP.

### Example 2

### Immobilization of trisodium salt of 8-hydroxypyrene-1,3,6-trisulfonate (HPTS)

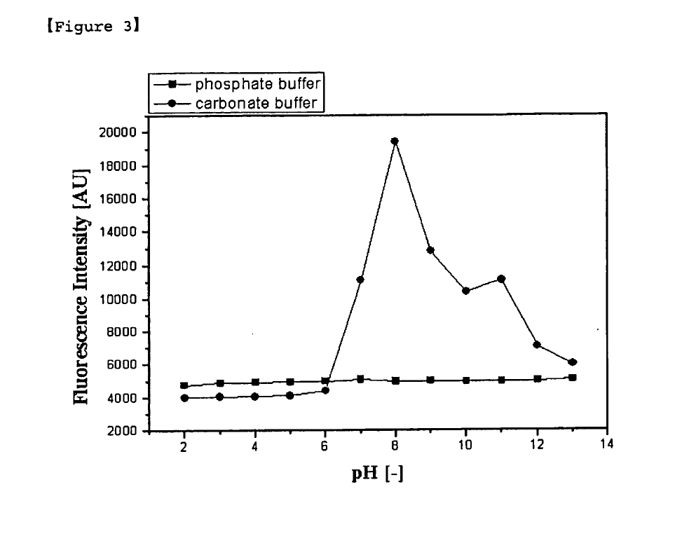

3-Glycidoxypropyltrimethoxysilane (GPTMS; 1.5 ml), methyltriethoxysilane (MTES; 1.5 ml), ethanol (6.95 ml) and 35% HCl (0.5 ml) were mixed together and stirred at room temperature for three days to induce a condensation reaction. To the sol-gel solution thus prepared (0.5 ml), 1 mM HPTS solution (0.5 ml) which had been dissolved in ethanol was added to give a HPTS mixture solution. 15 μL of this HPTS mixture solution was evenly coated onto the bottom surface of wells of a microtiter plate to prepare a fluorescent sensing membrane that can be used for detection of carbon dioxide. The sol-gel solution comprising coated HPTS was dried at room temperature for five days and further dried at 70 for two days for improving a mechanical strength and surface smoothness of thus-prepared HTPS gel. Thereafter, PMMA, a hydrophobic polymer, was coated to the top of the HPTS sensing membrane to prepare a HPTS sensing membrane that is coated with a hydrophobic polymer membrane. To measure the membrane's permeability selective to carbon dioxide, phosphate buffer solution (0.1M) was used as a standard pH buffer solution and carbonate buffer solution (0.1M) was used to change the concentration of carbon dioxide in an aqueous solution. Experiments were carried out under said condition to measure the membrane's permeability selective to carbon dioxide. The results are shown in FIG. 3. As a result, it was found that carbon dioxide gas which had been generated from the carbonate solution permeated into the hydrophobic membrane to react with HPTS comprised in the sensing membrane, resulting in a change in fluorescence intensity. On the other hand, hydrogen ions contained in the phosphate buffer solution could not permeate into the sensing membrane, thus not yielding any fluorescence. Therefore, it was confirmed that only carbon dioxide molecules could selectively permeate into the sensing membrane of the present invention.

### Example 3

### Optical determination of pH using 6-aminofluorescein

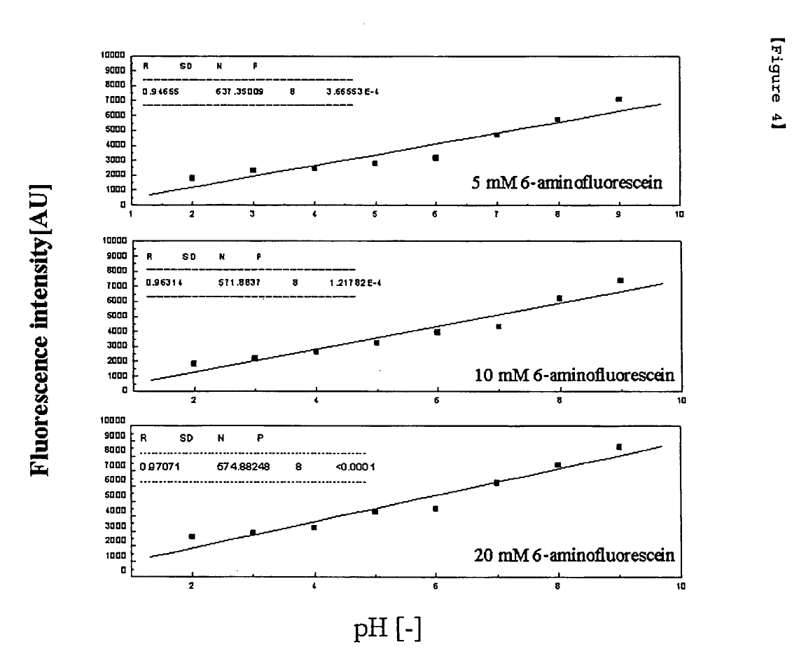

3-Glycidoxypropyltrimethoxysilane (GPTMS; 1.25 ml), methyltriethoxysilane (MTES; 2.5 ml), ethanol (6.2 ml) and 35% HCl (0.5 ml) were mixed together and stirred at room temperature for 16 hrs. To the sol-gel solution thus prepared (0.5 ml), 5 mM, 10 mM or 20 mM 6-aminofluorescein solution (0.5 ml) which had been dissolved in ethanol was added, and then thoroughly mixed using a stirrer and stored at room temperature for 2 hrs to give a 6-aminofluorescein mixture solution. Thus obtained mixture solution was spin-coated onto a polyproplylene film to prepare a sensing membrane. The resulting membrane was fixed to the bottom surface of a microtiter plate using a transparent two-sided adhesive tape. In order to measure a change in fluorescence intensity of said sensing membrane according to varying pH, the sensing membrane which comprises the fluorescent dye at different concentrations of 5, 10 and 20 mM prepared in phosphate buffer solution (0.1M) was titrated with 1N HCl and NaOH to change the pH. The results are shown in FIG. 4. As a result, it was found that the sensitivity of the sensing membrane remained almost the same for those prepared with the fluorescent dye at different concentrations of 5, 10 and 20 mM. In addition, precision degree of the sensing membrane increased with increasing concentration of the fluorescent dye (i.e., from 0.94655 to 0.97071).

### Example 4

### Preparation of a Sensing Membrane which Comprises a Bioconjugate

Cadmium acetate dehydrate (0.6 mM, 147 mg) and stearic acid (2.13 mM, 607 mg) were added to a 50 ml three-neck flask and heated at 150° C. under vacuum until a colorless solution was obtained. After cooling the mixture to room temperature, hexadecylamine (1.94 g) and trioctylphosphine oxide (TOPO; 2.2 g) was added to the flask and the resulting mixture was heated at 120-150° C. under vacuum. The reaction flask was then purged with nitrogen gas and heated to a temperature of 310-320° C., at which point 211 g of selenium that had been dissolved in trioctylphosphine (TOP; 2.5 ink) was quickly added to the flask while stirring the mixture vigorously. The mixture solution was heated for 25 sec before being removed from a heating mantle, and then cooled to room temperature. The reaction mixture was then dissolved in chloroform, followed by precipitation with methanol having the same volume to purify thus-obtained CdSe nanoparticles. In the next step, said purified CdSe particles were used for the synthesis of CdSe/ZnS core-shell QDs (CZ-QDs). A mixture of hexadecylamine (2 g) and TOPO (2.5 g) was added to a 50 in three-neck flask, degassed and heated at 180° C. The purified CdSe particles that had been dispersed in chloroform (2 ml) was added to the above mixture solution at 180° C. Chloroform was removed completely by using a pump and the flask was purged with nitrogen gas. The reaction temperature was raised to 180-185° C. A mixture comprising zinc acetate (54 mg) which had been dissolved in 1 ml TOP and hexamethyldisilathiane ((TMS)₂S; 0.05 ml) was added dropwise to the flask for 5-10 min, followed by the stirring at 180-185° C. for 1 hr. 200 mg of CZ-QDs in TOP-TOPO-hexadecylamine was dissolved in anhydrous chloroform and ethanol, respectively, and then purified by precipitation. Wet precipitates were dispersed in a mixture of 2 ml N,N-dimethylformamide (DMF) and 0.25 ml 3-mercaptopropionic acid. The mixture was sonicated for about 30 min until it becomes transparent. The resulting mixture was stored at room temperature for one week. For a next step, 0.5˜0.7 ml of 4-dimethylaminopyridine (DMAP) that had been dissolved in DMF (50 mg DMAP/1.0 ml DMF) was added to the mixture. Then the solution was centrifuged at 5000 rpm for 30 min. Supernatant was removed and the precipitates were dried in a desiccator to obtain CdSe/ZnS core-shell QDs coated with hydrophilic surfactants.

A mixture comprising GPTMS and MTES in a volume ratio of 1:2 (GM2) and a mixture comprising GPTMS and APTMS in a volume ratio of 4:1 (GA2) were separately mixed with 99% ethanol to prepare a sol-gel mixture. 35% HCl was added to each of these mixture solutions (40 μl/ml), followed by keeping the resulting solutions at room temperature at least for 2 hrs before they are used for a next step. To the sol-gel GM2 (200 μl), MPA-coated QDs (50 μl) produced in the above Preparation example 2 was added to prepare a transducer. A mixture comprising MPA-coated QDs and said sol-gel solution was thoroughly mixed by mechanical stirring, and then stored at room temperature for 2 hrs. The mixture of MPA-coated QDs (5 μl) was aliquoted to the bottom surface of a 96-well microtiter plate, and dried at 95° C. for 18 hrs. After the heat treatment, the sol-gel GA2 was added on top of the transducer and an enzyme solution (40 μl; GOD: 100 unit, LOD: 1 unit or TOD: 0.005 unit) was further added to the wells of a 96-well microtiter plate. The enzyme was immobilized for 18 hrs at room temperature.

### Example 5

### Immobilization of the Divided Optic Sensing Membranes

To a sol-gel solution (0.5 ml) obtained by mixing 3-glycidoxypropyltrimethoxysilane (GPTMS; 1.25 ml), methyltriethoxysilane (MTES; 2.5 ml), ethanol (6.2 ml) and 35% HCl (0.5 ml) and stirring them at room temperature for 16 hrs, 5 mg/mL RuDPP solution or 20 mM 6-aminofluorescein solution that had been prepared by dissolving them in ethanol (0.5 ml) were added, respectively, followed by a thorough mixing using a stirrer. After storing the resulting mixture at room temperature for 2 hrs, a mixture solution comprising each of said fluorescent dyes was prepared, which was then aliquoted into four divided sections of the well that had been created by a cross-shaped insert pushed down to the bottom surface of each well. The aliquoted solution was dried at room temperature for five days. After removing the cross-shaped insert, the solution was again dried at 70° C. for two days.

### Experimental example 1

### Monitoring of an E. Coli Fermentation Process

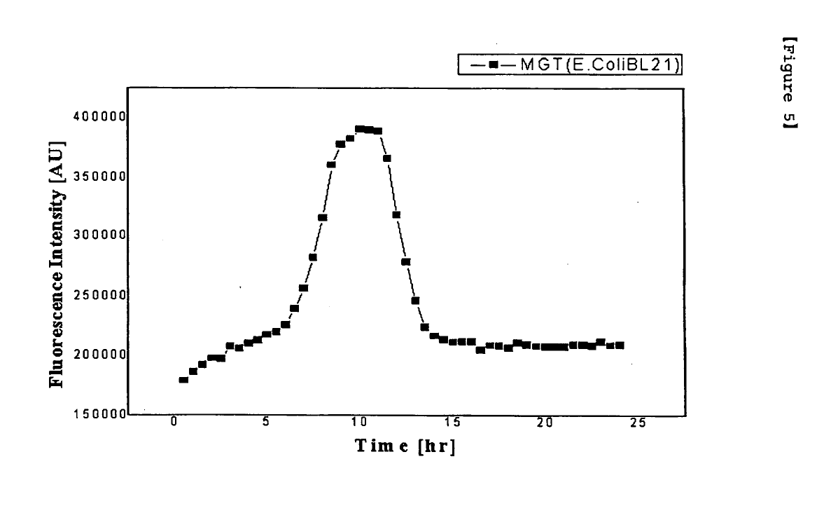

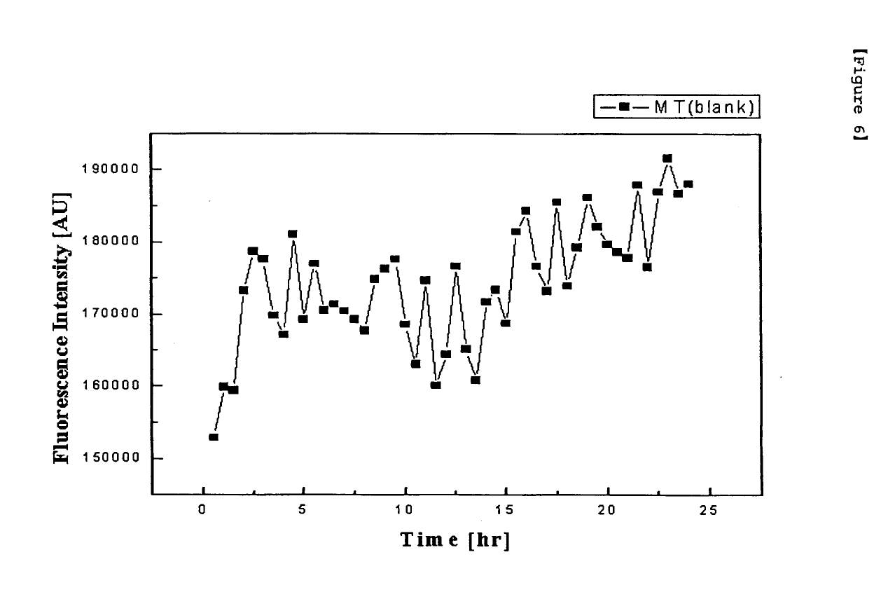

For monitoring a fermentation process using E. coliBL21, pre-culture was first carried out using LB media having composition of NaCl (10 g/L), tryptone (10 g/L), and yeast extract (5 g/L) in a shaker incubator. The main culture was carried out in 1.5 mL media (total volume) contained in a 24-well microtiter plate to which a membrane for sensing dissolved oxygen had been attached. The microorganism strain (1%) was inoculated to said microtiter plate and culturing was carried out. In order to monitor the progress of culture and the fluorescence, a microtiter plate reader (Victor 1420, Perkin Elmer, Finland) was used. A change in fluorescence intensity was monitored every thirty minutes during the culture and the results are shown in FIG. 5. It was found for E. coliBL21 that, at the beginning of the culture there was a certain period during which the cell growth stalled. However, as soon as the bacterial cells entered a logarithmic growth phase, oxygen was rapidly consumed by them, yielding a dramatic increase in fluorescence intensity. After the logarithmic growth phase was over, the fluorescence intensity decreased and then stayed at a constant level. For a control group in which the well was filled with blank culture media instead of culturing any bacterial cells, the results are shown in FIG. 6. As it can be seen from FIG. 6, the fluorescence intensity remained the same for 24 hrs.

### Experimental example 2

### Monitoring of a Yeast Fermentation Process

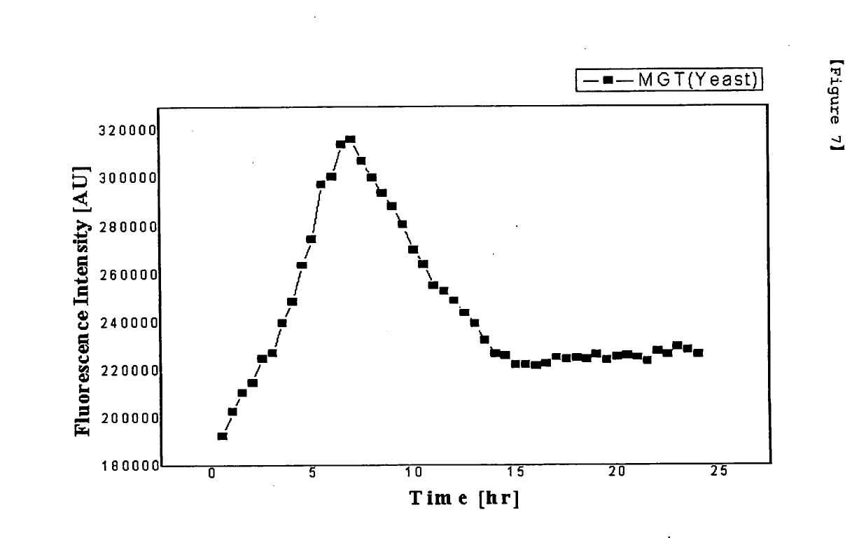

For monitoring a fermentation process using a yeast strain P. Pastoris X-33, pre-culture was first carried out using YPD media having composition of peptone (20 g/L), dextrose (20 g/L), and yeast extract (10 g/L) in a shaker incubator. The main culture was carried out in 1.5 mL media (total volume) contained in a 24-well microtiter plate to which a membrane for sensing dissolved oxygen had been attached. The microorganism strain (1%) was inoculated to said microtiter plate and culturing was carried out. In order to monitor the progress of culture and the fluorescence, a microtiter plate reader was used. A change in fluorescence intensity was monitored every thirty minutes during the culture and the results are shown in FIG. 7.

### Experimental Example 3

### Monitoring of a Bascillus Fermentation Process

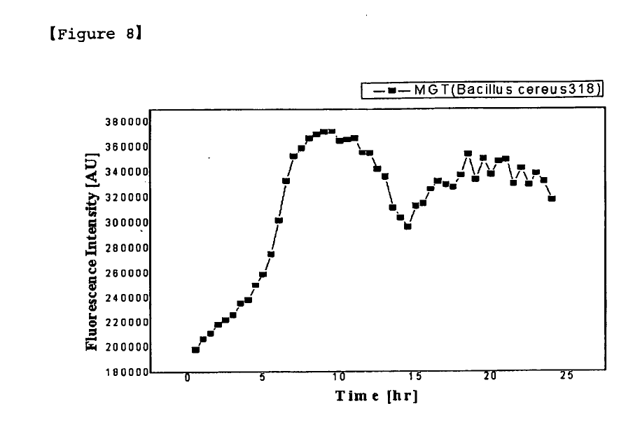

For monitoring a fermentation process using Bacillus cereus318, pre-culture was first carried out using a media exclusive for Bacillus cereus318 having composition of glucose (5 g/L), peptone (5 g/L), yeast extract (5 g/L) and NaHCO₃ (3 g/L) in a shaker incubator. The main culture was carried out in 1.5 mL media (total volume) contained in a 24-well microtiter plate to which a membrane for sensing dissolved oxygen had been attached. The microorganism strain (1%) was inoculated to said microtiter plate and culturing was carried out. In order to monitor the progress of culture and the fluorescence, a microtiter plate reader was used. A change in fluorescence intensity was monitored every thirty minutes during the culture and the results are shown in FIG. 8.

### INDUSTRIAL APPLICABILITY

The multi-channel micro bioreactor according to the present invention has a number of wells which can comprise various sensor materials that are independent to each other. Therefore, a number of optical detection results can be simultaneously obtained via multi-channel and on-line monitoring based on an in-situ detection method. In addition, the multi-channel micro bioreactor according to the present invention is advantageous in that research labor forces and cost can be saved compared to conventional bioreactors, thanks to its small size.

1. A multi-channel mini bioreactor having a number of wells in which an optical sensing membrane comprising a fluorescent dye and a bioconjugate, or a sensor material including a biomolecule conjugated to a fluorescent dye and a bioconjugate, is formed.

2. The multi-channel mini bioreactor of claim 1, wherein said wells are formed and aligned in a single frame.

3. The multi-channel mini bioreactor of claim 2, wherein the optical sensing membrane, which is a single membrane or divided into number n, is individually formed depending on each well, with n being an integer between 1 and 100.

4. The multi-channel mini bioreactor of claim 3, wherein said optical sensing membrane contains a single or more than one sensor material, that is adsorbed, bonded covalently, or captured by nano particles and micro particles.

5. The multi-channel mini bioreactor of claim 4, wherein said optical sensing membrane is formed at the bottom of a well.

6. The multi-channel mini bioreactor of claim 1, wherein said fluorescent dye is selected from the group consisting of a ruthenium complex, HPTS (8-hydroxypyrene-1,3,6-trisulfonate trisodium salt) and fluorescein amine.

7. The multi-channel mini bioreactor of claim 6, wherein said fluorescent dye detects dissolved oxygen, carbon dioxide and pH optically.

8. The multi-channel mini bioreactor of claim 6, wherein the hydrophobic polymer membrane is coated on top of the optical sensing membrane comprising said HPTS in order to achieve a selective detection of dissolved carbon dioxide.

9. The multi-channel mini bioreactor of claim 8, wherein said hydrophobic polymer membrane is selected from the group consisting of a silicone resin, PMMA and vernice, or a mixture thereof.

10. The multi-channel mini bioreactor of claim 1, wherein quantum dots having a CdSe core that is coated with a ZnS shell are coated with a hydrophilic surfactant and then conjugated with oxidases or oxidases and peroxidases in said bioconjugate.

11. The multi-channel mini bioreactor of claim 10, wherein said oxidases are selected from the group consisting of glucose oxidase, lactate oxidase, tyramine oxidase, ascorbic acid oxidase, and xanthine oxidase, or a mixture thereof.

12. The multi-channel mini bioreactor of claim 1, wherein said biomolecule is protein, amino acid, enzyme, antigen and antibody.

13. The multi-channel mini bioreactor of claim 1, wherein said optical sensing membrane immobilizes the sensor material by using a sol-gel method.

14. The multi-channel mini bioreactor of claim 13, wherein said sol-gel method immobilizes the sensor material by using alkoxysilane selected from the group consisting of 3-glycidoxypropyltrimethoxysilane (GPTMS), methyltriethoxysilane (MTES), aminopropyltrimethoxysilane (APTMS), phenyltrimethoxysilane (PTMS), and methyltrimethoxysilane (MTMS), or a mixture thereof.

15. The multi-channel mini bioreactor of claim 1, wherein a baffle board is installed on the cover of said wells.

16. The multi-channel mini bioreactor of claim 15, wherein said baffle board increases a stirring effect, an oxygen-transferring speed and a material-transferring speed.

17. The multi-channel mini bioreactor of claim 1, wherein a transparent polymer film coated with an optical sensing membrane is immobilized on said wells using an adhesive.

18. The multi-channel mini bioreactor of claim 17, wherein said optical sensing membrane can be divided into n sections and each divided membrane can achieve an individual optical detection, with n being an integer between 1 and 100.

19. A multi-channel optical detection system prepared by using the multi-channel mini bioreactor of claim 1.
